(19) United States
(12) Patent Application Publication
(10) Pub. No.: US 2026/0215875 A1
(43) Pub. Date: Jul. 30, 2026

(54) DEPTH-BASED GENERATION OF MIXED-REALITY IMAGES

(71) Applicant: Intuitive Surgical Operations, Inc., Sunnyvale, CA (US)

(72) Inventors: Pourya Shirazian, Menlo Park, CA (US); A. Jonathan McLeod, Sunnyvale, CA (US); Hidenori Shikata, Saratoga, CA (US)

(21) Appl. No.: 19/146,457

(22) PCT Filed: Mar. 6, 2024
(86) PCT No.: PCT/US2024/018620

§ 371 Date: Jul. 8, 2025

Related U.S. Application Data
(60) Provisional application No. 63/450,240, filed on Mar. 6, 2023.

(51) Int. Cl. A61B 90/00; A61B 34/10; A61B 34/30; G06T 7/00; G06T 7/50; G06V 10/25

(52) U.S. Cl. CPC A61B 90/36; A61B 34/10; A61B 34/30; G06T 7/0012; G06T 7/50; G06V 10/25; A61B 2034/105; A61B 2090/365; G06T 2207/10028; G06T 2207/20104

(57) ABSTRACT

An illustrative system may be configured to determine a region of interest within an image depicting a scene that includes one or more anatomical objects, access a virtual model associated with the one or more anatomical objects, and present a combined image that includes the virtual model superimposed on the image. The combined image may be presented by directing a display device to display a first portion of the virtual model that is positioned within the region of interest of the image and abstain from displaying a second portion of the virtual model that is positioned outside the region of interest of the image.

### RELATED APPLICATIONS

This application claims priority to U.S. Provisional Patent Application No. 63/450,240, filed Mar. 6, 2023, the contents of which is hereby incorporated by reference in its entirety.

### BACKGROUND INFORMATION

During a medical procedure, such as a minimally-invasive medical procedure, an imaging device (e.g., a stereoscopic imaging device) may be used to capture images of internal anatomy within a subject. In some instances, it may be desirable to visualize one or more portions of the internal anatomy that may be obstructed from view of the imaging device. For example, it may be desirable to visualize a feature (e.g., vasculature, etc.) of the internal anatomy that may be located beneath a surface of the internal anatomy depicted in the images. In some scenarios, a virtual model including (i.e., depicting) the one or more portions of the internal anatomy may be shown in combination with the captured images such as to provide a visualization of the one or more portions of the internal anatomy that may be obstructed.

However, the virtual model may, in some instances, include portions of the internal anatomy in addition to the one or more portions of the internal anatomy that may be obstructed. As such, the virtual model may clutter a view of the internal anatomy in the captured images. Moreover, the combination of the virtual model and the captured images may, in some instances, provide a poor depth perception of the one or more portions of the internal anatomy between the virtual model and the captured images.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its purpose is to present one or more aspects of the systems and methods described herein as a prelude to the detailed description that is presented below.

An illustrative computer-assisted medical system includes a repositionable manipulator arm configured to be coupled to an imaging device that may be configured to capture an image depicting a scene that includes one or more anatomical objects positioned within a field of view of the imaging device and a computing device communicatively coupled with the imaging device. The computing device may be configured to determine a region of interest within the image, access a virtual model associated with the one or more anatomical objects, and present a combined image that includes the virtual model superimposed on the image by directing a display device to display, within the combined image, a first portion of the virtual model that is positioned within the region of interest of the image and abstain from displaying, within the combined image, a second portion of the virtual model that is positioned outside the region of interest of the image.

An illustrative system includes a memory storing instructions and one or more processors communicatively coupled to the memory. The one or more processors may be configured to execute the instructions to perform a process comprising determining a region of interest within an image depicting a scene that includes one or more anatomical objects, accessing a virtual model associated with the one or more anatomical objects, and presenting a combined image that includes the virtual model superimposed on the image. The presenting the combined image may include directing a display device to display, within the combined image, a first portion of the virtual model that is positioned within the region of interest of the image and abstain from displaying, within the combined image, a second portion of the virtual model that is positioned outside the region of interest of the image.

An illustrative method includes determining a region of interest within an image depicting a scene that includes one or more anatomical objects, accessing a virtual model associated with the one or more anatomical objects, and presenting a combined image that includes the virtual model superimposed on the image. The presenting the combined image may include directing a display device to display, within the combined image, a first portion of the virtual model that is positioned within the region of interest of the image and abstain from displaying, within the combined image, a second portion of the virtual model that is positioned outside the region of interest of the image.

An illustrative non-transitory computer-readable medium may store instructions that, when executed, direct a processor of a computing device to perform a process comprising determining a region of interest within an image depicting a scene that includes one or more anatomical objects, accessing a virtual model associated with the one or more anatomical objects, and presenting a combined image that includes the virtual model superimposed on the image. The presenting the combined image may include directing a display device to display, within the combined image, a first portion of the virtual model that is positioned within the region of interest of the image and abstain from displaying, within the combined image, a second portion of the virtual model that is positioned outside the region of interest of the image.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an illustrative computer-assisted medical system.

FIG. 2 shows an illustrative implementation including a mixed-reality image generation system.

FIG. 3 shows an illustrative method of operating a mixed-reality image generation system.

FIGS. 4A-4C show illustrative implementations of displays that may be generated using a mixed-reality image generation system.

FIGS. 5A and 5B show illustrative implementations of determining a region of interest using a mixed-reality image generation system.

FIGS. 6A-6D show further illustrative implementations of determining a region of interest using a mixed-reality image generation system.

FIG. 7 shows an illustrative method of operating a mixed-reality image generation system.

FIGS. 8A and 8B show illustrative implementations of adjusting a visual characteristic using a mixed-reality image generation system.

FIG. 9 shows an illustrative implementation of adjusting a visual characteristic using a mixed-reality image generation system.

FIGS. 10A and 10B show illustrative implementations of adjusting a visual characteristic using a mixed-reality image generation system.

FIG. 11 shows an illustrative computing system according to principles described herein.

### DETAILED DESCRIPTION

An illustrative system for producing mixed-reality images may be configured to confine an overlay of a virtual model to a region of interest within an image. For example, a mixed-reality image generation system may be configured to determine a region of interest within an image depicting a scene that includes one or more anatomical objects, access a virtual model of the one or more anatomical objects, and present a combined image that includes the virtual model superimposed on the image by directing a display device to display, within the combined image, a first portion of the virtual model that is positioned within the region of interest of the image and abstain from displaying, within the combined image, a second portion of the virtual model that is positioned outside the region of interest of the image.

In some implementations, one or more regions of interest may be determined in the image by the mixed-reality image generation system, such as based on a depth of the one or more anatomical objects. Additionally or alternatively, one or more visual characteristics (e.g., transparency, brightness, etc.) of the virtual model may be scaled by the mixed-reality image generation system, such as based on the depth of the one or more anatomical objects.

The principles described herein may result in improved mixed-reality images compared to conventional techniques that do not confine an overlay of the virtual model to the region of interest, as well as provide other benefits as described herein. For example, confining the overlay of the virtual model to the region of interest may allow the scene to be depicted more clearly, such as by depicting one or more features of the anatomical object that may be included in the virtual model (e.g., beneath a surface of the anatomical object) within the region of interest without obstructing other portions of the scene depicted in the image outside of the region of interest. This may allow a medical procedure associated with the anatomical object to be performed more quickly and/or easily. Additionally, confining the overlay of the virtual model to the region of interest may improve a depth perception of the anatomical object between the virtual model and the scene depicted in the image.

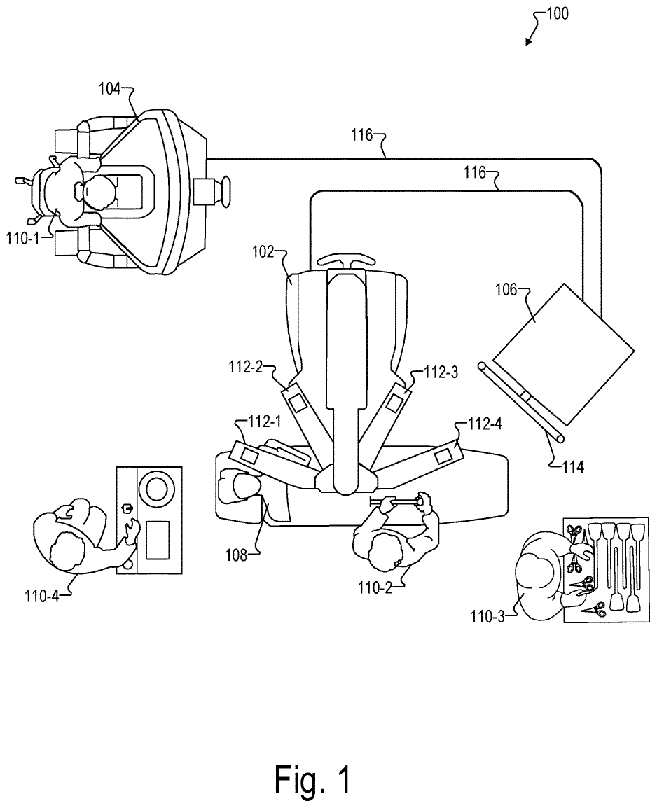

In some examples, one or more components of a system for producing mixed-reality images may be implemented by a computer-assisted medical system. For example, FIG. 1 shows an illustrative computer-assisted medical system 100 that may be used to perform various types of medical procedures including surgical and/or non-surgical procedures.

As shown, computer-assisted medical system 100 may include a manipulator assembly 102 (a manipulator cart is shown in FIG. 1), a user control apparatus 104, and an auxiliary apparatus 106, all of which are communicatively coupled to each other. Computer-assisted medical system 100 may be utilized by a medical team to perform a computer-assisted medical procedure or other similar operation on a body of a patient 108 or on any other body as may serve a particular implementation. As shown, the medical team may include a first user 110-1 (such as a surgeon for a surgical procedure), a second user 110-2 (such as a patient-side assistant), a third user 110-3 (such as another assistant, a nurse, a trainee, etc.), and a fourth user 110-4 (such as an anesthesiologist for a surgical procedure), all of whom may be collectively referred to as users 110, and each of whom may control, interact with, or otherwise be a user of computer-assisted medical system 100. More, fewer, or alternative users may be present during a medical procedure as may serve a particular implementation. For example, team composition for different medical procedures, or for non-medical procedures, may differ and include users with different roles.

While FIG. 1 illustrates an ongoing minimally invasive medical procedure such as a minimally invasive surgical procedure, it will be understood that computer-assisted medical system 100 may similarly be used to perform open medical procedures or other types of operations. For example, operations such as exploratory imaging operations, mock medical procedures used for training purposes, and/or other operations may also be performed.

As shown in FIG. 1, manipulator assembly 102 may include one or more manipulator arms 112 (e.g., manipulator arms 112-1 through 112-4) to which one or more instruments may be coupled. The instruments may be used for a computer-assisted medical procedure on patient 108 (e.g., in a surgical example, by being at least partially inserted into patient 108 and manipulated within patient 108). While manipulator assembly 102 is depicted and described herein as including four manipulator arms 112, it will be recognized that manipulator assembly 102 may include a single manipulator arm 112 or any other number of manipulator arms as may serve a particular implementation. While the example of FIG. 1 illustrates manipulator arms 112 as being robotic manipulator arms, it will be understood that, in some examples, one or more instruments may be partially or entirely manually controlled, such as by being handheld and controlled manually by a person. For instance, these partially or entirely manually controlled instruments may be used in conjunction with, or as an alternative to, computer-assisted instrumentation that is coupled to manipulator arms 112 shown in FIG. 1.

During the medical operation, user control apparatus 104 may be configured to facilitate teleoperational control by user 110-1 of manipulator arms 112 and instruments attached to manipulator arms 112. To this end, user control apparatus 104 may provide user 110-1 with imagery of an operational area associated with patient 108 as captured by an imaging device. Manipulator arms 112 or any instruments coupled to manipulator arms 112 may mimic the dexterity of the hand, wrist, and fingers of user 110-1 across multiple degrees of freedom of motion. In this manner, user 110-1 may intuitively perform a procedure using one or more of manipulator arms 112 or any instruments coupled to manipulator arms 112. in order to perform one or more surgical procedures (e.g., an incision procedure, a suturing procedure, etc.).

Auxiliary apparatus 106 may include one or more computing devices configured to perform auxiliary functions in support of the medical procedure, such as providing insufflation, electrocautery energy, illumination or other energy for imaging devices, image processing, or coordinating components of computer-assisted medical system 100. In some examples, auxiliary apparatus 106 may be configured with a display monitor 114 configured to display one or more user interfaces, or graphical or textual information in support of the medical procedure. In some instances, display monitor 114 may be implemented by a touchscreen display and provide user input functionality. Augmented content provided by a region-based augmentation system may be similar, or differ from, content associated with display monitor 114 or one or more display devices in the operation area (not shown).

Manipulator assembly 102, user control apparatus 104, and auxiliary apparatus 106 may be communicatively coupled one to another in any suitable manner. For example, as shown in FIG. 1, manipulator assembly 102, user control apparatus 104, and auxiliary apparatus 106 may be communicatively coupled by way of control lines 116, which may represent any wired or wireless communication link as may serve a particular implementation. To this end, manipulator assembly 102, user control apparatus 104, and auxiliary apparatus 106 may each include one or more wired or wireless communication interfaces, such as one or more local area network interfaces, Wi-Fi network interfaces, cellular interfaces, and so forth.

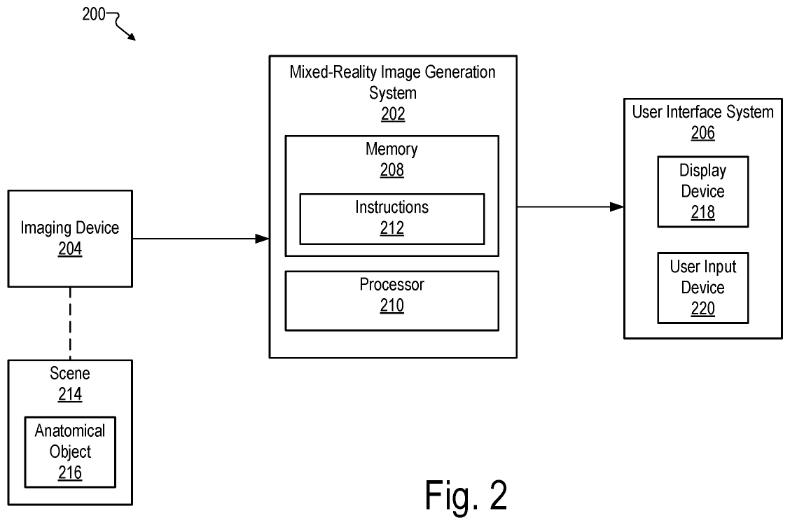

FIG. 2 shows an illustrative implementation 200 configured to generate a mixed-reality image. As shown, implementation 200 includes a mixed-reality image generation system 202 in communication with an imaging device 204 and a user interface system 206. Implementation 200 may include additional or alternative components as may serve a particular implementation. In some examples, implementation 200 or certain components of implementation 200 may be implemented by a computer-assisted medical system, such as computer-assisted medical system 100 discussed above.

Mixed-reality image generation system 202 may be implemented by one or more computing devices and/or computer resources (e.g., processors, memory devices, storage devices, etc.) as may serve a particular implementation. As shown, mixed-reality image generation system 202 may include, without limitation, a memory 208 and a processor 210 selectively and communicatively coupled to one another. Memory 208 and processor 210 may each include or be implemented by computer hardware that is configured to store and/or process computer software. Various other components of computer hardware and/or software not explicitly shown in FIG. 2 may also be included within mixed-reality image generation system 202. In some examples, memory 208 and/or processor 210 may be distributed between multiple devices and/or multiple locations as may serve a particular implementation.

Memory 208 may store and/or otherwise maintain executable data used by processor 210 to perform any of the functionality described herein. For example, memory 208 may store instructions 212 that may be executed by processor 210. Memory 208 may be implemented by one or more memory or storage devices, including any memory or storage devices described herein, that are configured to store data in a transitory or non-transitory manner. Instructions 212 may be executed by processor 210 to cause mixed-reality image generation system 202 to perform any of the functionality described herein. Instructions 212 may be implemented by any suitable application, software, code, and/or other executable data instance. Additionally, memory 208 may also maintain any other data accessed, managed, used, and/or transmitted by processor 210 in a particular implementation.

Processor 210 may be implemented by one or more computer processing devices, including general purpose processors (e.g., central processing units (CPUs), graphics processing units (GPUs), microprocessors, etc.), special purpose processors (e.g., application specific integrated circuits (ASICs), field-programmable gate arrays (FPGAs), etc.), image signal processors, or the like. Using processor 210 (e.g., when processor 210 is directed to perform operations represented by instructions 212 stored in memory 208), mixed-reality image generation system 202 may perform various operations as described herein.

Imaging device 204 may be implemented by a stereoscopic imaging device or other suitable device configured to capture and output one or more images (e.g., images, videos, a sequence of image frames, etc.) depicting a scene 214. In some implementations, imaging device 204 may include, but is not limited to, one or more of: video imaging devices, infrared imaging devices, visible light imaging devices, non-visible light imaging devices, intensity imaging devices (e.g., color, grayscale, black and white imaging devices), depth imaging devices (e.g., stereoscopic imaging devices, time-of-flight imaging devices, infrared imaging devices, red-green-blue (RGB) imaging devices, red-green-blue and depth (RGB-D) imaging devices, light detection and ranging (LIDAR) imaging devices, etc.).

In some implementations, the images captured by imaging device 204 may include image data (e.g., color, grayscale, saturation, intensity, brightness, depth, etc.). The image data may, in some instances, be associated with data points expressed in a common coordinate frame such as 3D voxels or two-dimensional (2D) pixels of images captured by imaging device 204. In some implementations, imaging device 204 may be moved relative to scene 214 to capture one or more images of scene 214 at different viewpoints.

Scene 214 may include an environment (e.g., an area within a subject of a medical procedure) and/or one or more objects within an environment. In some examples, scene 214 may include a surgical area associated with a body on or within which the medical procedure is being performed (e.g., a body of a live animal, a human or animal cadaver, a portion of human or animal anatomy, tissue removed from human or animal anatomies, non-tissue work pieces, training models, etc.). For example, scene 214 may include one or more anatomical objects 216 positioned within a field of view of imaging device 204. Anatomical object 216 may include an object associated with a subject (e.g., a body of a live animal, a human or animal cadaver, a portion of human or animal anatomy, tissue removed from human or animal anatomies, non-tissue work pieces, training models, etc.). In some implementations, anatomical object 216 may include tissue of a subject (e.g., an organ, soft tissue, connective tissue, etc.). Still other non-anatomical objects may be included within scene 214, such as physical tools (e.g., scalpels, scissors, forceps, clamps, etc.) and/or other objects (e.g., staples, mesh, sponges, etc.) used for a medical procedure. In certain embodiments, scene 2014 may include a portion of an anatomical object rather than its entirety.

In some implementations, a repositionable manipulator arm (e.g., manipulator arms 112 of computer-assisted medical system 100) may be coupled to imaging device 204. For example, the repositionable manipulator arm may be movable to position the field of view of imaging device 204 to include anatomical object 216. In some implementations, an additional repositionable manipulator arm (e.g., manipulator arms 112 of computer-assisted medical system 100) may be coupled to an instrument such that the additional repositionable manipulator arm may be movable to position the instrument within the field of view of imaging device 204 (e.g., to manipulate anatomical object 216).

User interface system 206 of the illustrated implementation comprises a display device 218 and a user input device 220. Display device 218 may be implemented by a monitor or other suitable device configured to display information to a user. For example, display device 218 may be configured to display one or more images captured by imaging device 204 and/or mixed-reality images generated by mixed-reality image generation system 202. User input device 220 may be implemented by any suitable device or devices (e.g., a button, joystick, touchscreen, keyboard, handle, microphone, etc.) configured to receive a user input, for example, to interact with the display presented by display device 218 and/or cause movement of one or more repositionable manipulator arms.

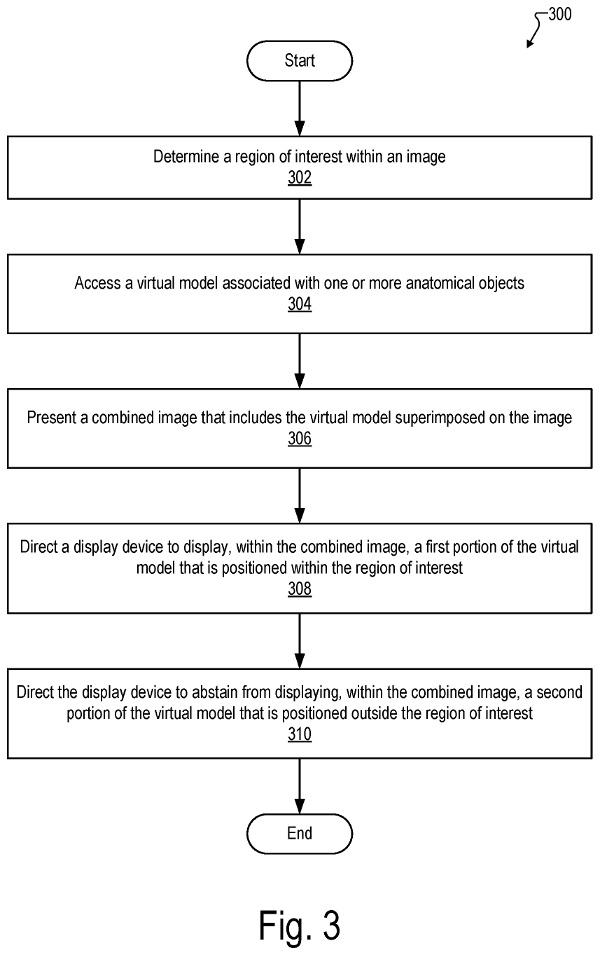

FIG. 3 shows an illustrative method 300 that may be performed by mixed-reality image generation system 202. While FIG. 3 illustrates exemplary operations according to one embodiment, other embodiments may omit, add to, reorder, and/or modify any of the operations shown in FIG. 3. Moreover, each of the operations depicted in FIG. 3 may be performed in any of the ways described herein.

As shown, mixed-reality image generation system 202 may, at operation 302, determine a region of interest within an image. In some implementations, mixed-reality image generation system 202 may determine the region of interest within the image by identifying an area (e.g., an area of interest) within at least a portion of the image. For example, the region of interest may include an area in the image depicting one or more anatomical objects (e.g., anatomical objects 216) of interest (e.g., associated with a medical procedure). To illustrate, the region of interest may encompass at least a portion of the one or more anatomical objects depicted in the image (e.g., the region of interest may encompass an entirety of a single anatomical object, an entirety of multiple anatomical objects, an anatomical object positioned within another anatomical object, a feature of an anatomical object, a portion of an anatomical object, a portion of multiple anatomical objects, etc.). In some implementations, the region of interest may include one or more anatomical objects associated with a medical procedure such that it may be desirable to visualize features of the one or more anatomical objects (e.g., beneath a surface depicted in the image). Moreover, the region of interest, in some instances, may have a specified shape (e.g., a circle, a square, freeform, etc.).

In some implementations, mixed-reality image generation system 202 may determine the region of interest by detecting a user input designating the region of interest. For example, a user may interact with a display of the image (e.g., using user input device 220) to designate the region of interest in the image.

Additionally or alternatively, mixed-reality image generation system 202 may determine the region of interest by identifying one or more anatomical objects in the image and designating an area in the image that depicts at least a portion of the one or more anatomical objects as the region of interest. For example, mixed-reality image generation system 202 may implement and apply artificial intelligence algorithms, such as machine learning algorithms, to identify one or more anatomical objects in the image and/or designate that region of interest as an area in the image that depicts at least a portion of the one or more anatomical objects. To illustrate, a machine learning algorithm may be used to identify one or more anatomical objects and/or features of one or more anatomical objects in the image such that mixed-reality image generation system 202 may determine the region of interest associated with the identified anatomical objects and/or features. Any suitable form of artificial intelligence and/or machine learning may be used, including, for example, deep learning, neural networks, etc. For example, a machine learning algorithm may be generated through machine learning procedures and applied to identification operations. In some implementations, the machine learning algorithm may be directed to identifying one or more anatomical objects and/or a feature of the one or more anatomical objects within the image. The machine learning algorithm may operate as an identification function that is applied to individual and/or fused imagery to classify the one or more anatomical objects in the image.

Still other suitable methods may be used to identify the one or more anatomical objects for determining the region of interest in addition to or instead of machine learning algorithms. For example, mixed-reality image generation system 202 may be configured to determine the region of interest by implementing and applying object recognition algorithms. For example, an object recognition algorithm may be used to identify objects (e.g., anatomical object 216) of predetermined types within the image, such as by comparing image data of the image to model object data of predetermined types of objects. Such model object data may be stored within a model database that may be communicatively coupled with mixed-reality image generation system 202.

In some implementations, the determining the region of interest may further include accessing the image. For example, mixed-reality image generation system 202 may access the image (e.g., captured by imaging device 204) depicting the one or more anatomical objects in any suitable manner. For example, mixed-reality image generation system 202 may access data representative of the image by way of one or more networks (e.g., a local area network, the Internet, etc.), directly from a computing device storing the image, directly from an imaging device (e.g., imaging device 204) configured to capture the images, etc.

In some implementations, the determining the region of interest may further include processing the image. For example, mixed-reality image generation system 202 may be configured to fuse or otherwise combine the image with another image depicting the one or more anatomical objects, such as by stitching non-overlapping voxels or pixels together (e.g., stitching images together along non-overlapping boundaries of the images), merging aligned and/or overlapping voxels or pixels (e.g., blending intensity and/or depth values for aligned voxels or pixels), etc.

Mixed-reality image generation system 202 may further, at operation 304, access a virtual model of the one or more anatomical objects. For example, the virtual model may include the one or more anatomical objects depicted within the region of interest in the image. In some implementations, the virtual model may represent the one or more anatomical objects and/or features of the one or more anatomical objects (e.g., beneath a surface depicted in the image) in one or more forms (e.g., solid, wireframe, surface, etc.). Mixed-reality image generation system 202 may access the virtual model in any suitable manner. For example, mixed-reality image generation system 202 may access data representative of the virtual model by way of one or more networks (e.g., a local area network, the Internet, etc.) directly from a computing device storing the virtual model. Additionally or alternatively, the data representative of the virtual model may be stored by mixed-reality image generation system 202 (e.g., by memory 208).

Mixed-reality image generation system 202 may further, at operation 306, present a combined image (e.g., a mixed-reality image) that includes the virtual model superimposed on the image. For example, presenting the combined image may include directing a display device (e.g., display device 218) to display the combined image. In some implementations, the virtual model may be confined to the region of interest such that mixed-reality image generation system 202 may, at operation 308, direct the display device to display, within the combined image, a first portion of the virtual model that is positioned within the region of interest of the image and, at operation 310, direct the display device to abstain from displaying, within the combined image, a second portion of the virtual model that is positioned outside the region of interest of the image. To illustrate, mixed-reality image generation system 202 may cause the virtual model to be superimposed on pixels or voxels of the image identified as being within the region of interest and abstain from superimposing the virtual model on pixels or voxels of the image identified as being outside the region of interest.

In some implementations, the virtual model may be repositioned in the combined image. For example, a user may interact with the display of the combined image (e.g., using user input device 220) to reposition the virtual model relative to the region of interest. Mixed-reality image generation system 202 may detect that the virtual model has been repositioned such that the presenting the combined image may include detecting that the first portion of the virtual model is repositioned to be outside the region of interest, and in response, abstaining from displaying the first portion. Additionally or alternatively, the presenting the combined image may include detecting that the second portion of the virtual model is repositioned to be inside the region of interest, and in response, displaying the second portion.

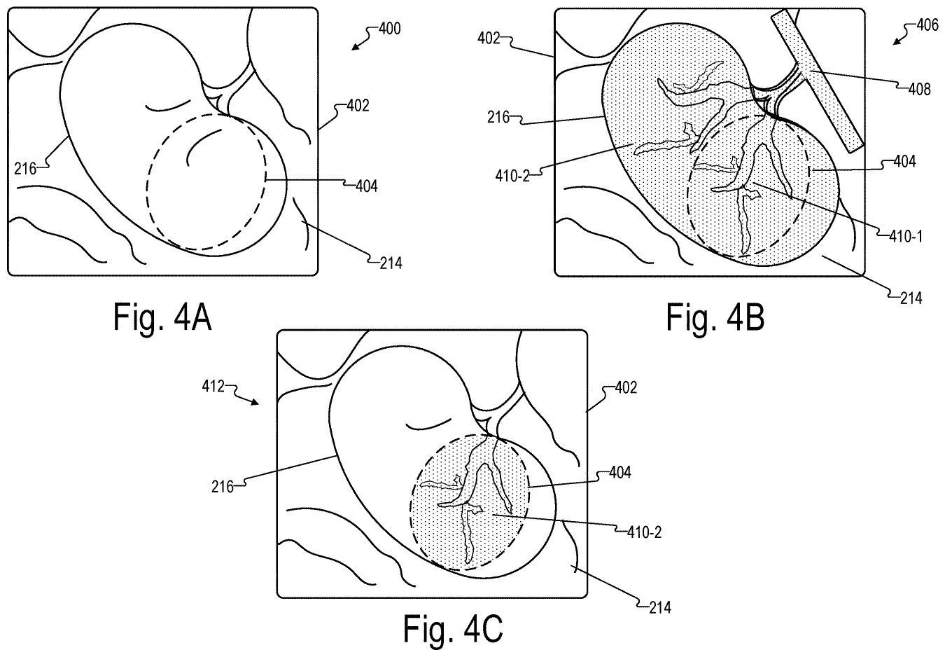

As illustrative examples, FIGS. 4A-4C show implementations of displays that may be generated using a mixed-reality image generation system 202. As shown, FIG. 4A shows an illustrative implementation 400 of a display of an image 402 (e.g., captured by imaging device 204) depicting a scene 214 including an anatomical object 216 (e.g., a kidney). Image 402 further includes a region of interest 404 (e.g., determined by mixed-reality image generation system 202 in any of the ways described herein). As shown, region of interest 404 encompasses a portion of anatomical object 216. For example, it may be desirable to visualize features of anatomical object 216 and/or other anatomical objects beneath an exterior surface of anatomical object 216 within the portion of anatomical object 216 indicated by region of interest 404 (e.g., to perform a medical procedure).

FIG. 4B shows an illustrative implementation 406 of a display of a virtual model 408 (e.g., accessed by mixed-reality image generation system 202) in combination with image 402. For example, virtual model 408 may include a 2D or 3D model of anatomical object 216 depicted in image 402. In some implementations, virtual model 408 may depict features (e.g., vasculature, etc.) of anatomical object 216 and/or other anatomical objects beneath the exterior surface of anatomical object 216 depicted in image 402. Additionally, virtual model 408 may, in some instances, include features and/or anatomical objects in addition to anatomical object 216 depicted in image 402.

In some implementations, virtual model 408 may be generated, such as based on preoperative imagery of anatomical object 216. For example, virtual model 408 may be generated based on a 3D image representation of anatomical object 216 from a scanning system (e.g., a Computerized Tomography (CT), a Magnetic Resonance Imaging (MRI), an Ultrasound, etc.). The 3D image representation of anatomical object 216 may include image data (e.g., pixels or voxels) arranged in a 3D grid configuration. Virtual model 408 may be derived from the 3D image representation of anatomical object 216, such as by generating vertices of virtual model 408 arranged in a 3D grid configuration and associating the vertices with 3D locations that correspond to locations of the image data within the 3D image representation of anatomical object 216.

In some implementations, virtual model 408 may be sized and/or oriented to correspond to anatomical object 216 depicted in image 402. To illustrate, a user may interact with the display of image 402 (e.g., using user input device 220) to position virtual model 408 within image 402. As shown, virtual model 408 includes a first portion 410-1 positioned within region of interest 404 and a second portion 410-2 extending beyond region of interest 404. In the illustrated example, the second portion 410-2 of virtual model 408 may obstruct portions of image 402 positioned outside region of interest 404.

FIG. 4C shows an illustrative implementation 412 of a display of virtual model 408 being confined to region of interest 404 in image 402. For example, mixed-reality image generation system 202 may direct the display device to display the first portion 410-1 of virtual model 408 within region of interest 404 and to abstain from displaying the second portion 410-2 of virtual model 408 outside region of interest 404. This may allow virtual model 408 to depict features of anatomical object 216 and/or other anatomical objects beneath an exterior surface of anatomical object 216 within region of interest 404 while not obstructing image 402 outside of region of interest 404. In some implementations, the displaying the first portion 410-1 of virtual model 408 may include displaying a boundary (e.g., an outline) of region of interest 404, such as to indicate a region of the first portion 410-1 of virtual model 408 being displayed. In instances where virtual model 408 is repositioned in image 402, mixed-reality image generation system 202 may abstain from displaying areas of the first portion 410-1 of virtual model 408 repositioned outside of region of interest 404 and/or display areas of the second portion 410-2 of virtual model 408 repositioned within region of interest 404.

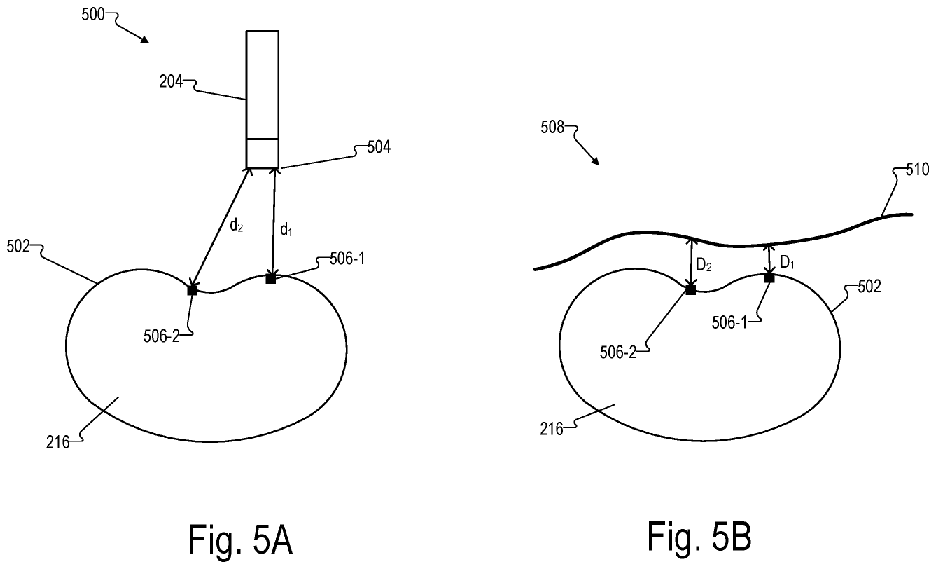

Various techniques may be used by mixed-reality image generation system 202 to determine region of interest 404. For example, FIG. 5A shows an illustrative implementation 500 of determining the region of interest based on a depth of anatomical object 216 (e.g., a physical depth based on the physical anatomical object 216 and/or a virtual depth based on the virtual anatomical object 216 depicted in the image) such as relative to imaging device 204. As shown, anatomical object 216 includes an exterior surface 502 that may be positioned at various depths relative to a distal end 504 (e.g., an end positioned toward anatomical object 216) of imaging device 204. The depths d of anatomical object 216 in the image (e.g., image 402) may be determined by accessing and/or generating a depth map of the scene (e.g., scene 214) captured by imaging device 204 including anatomical object 216. In some implementations, the depth map may be generated such as by processing stereoscopic images (e.g., using machine learning algorithms), using a simultaneous localization and mapping (SLAM) algorithm, and/or by a depth sensor (e.g., a time of flight sensor) associated with imaging device 204. Mixed-reality image generation system 202 may further identify anatomical object 216 (e.g., using machine learning algorithms, object identification, etc.) in the scene such as to generate the depth map.

To illustrate, mixed-reality image generation system 202 may generate a point cloud in the image having a plurality of nodes representative of surface points 506 (e.g., surface points 506-1 to 506-2) on exterior surface 502 of anatomical object 216. In some implementations, each node may be associated with an area in the image including one or more pixels or voxels. For illustrative purposes, FIG. 5A shows a first surface point 506-1 that may be associated with a first node in the image and a second surface point 506-2 that may be associated with a second node in the image. Additionally or alternatively, the nodes may be associated with one or more other points (e.g., a centroid, a closest point to imaging device 204, a farthest point to imaging device 204, etc.) of one or more anatomical objects 216 depicted in the image.

Mixed-reality image generation system 202 may determine a depth value associated with each node representative of a depth d (e.g., depths d₁ to d₃) of the respective surface point 506 relative to distal end 504 of imaging device 204. For example, the first node may be associated with a first depth value representative of a first depth d₁ of first surface point 506-1 relative to distal end 504 of imaging device 204 and the second node may be associated with a second depth value representative of a second depth d₂ of second surface point 506-2 relative to distal end 504 of imaging device 204. The depth values may be represented by any suitable metric, such as a discrete value (e.g., a distance, a ratio, a percentage, etc.).

Mixed-reality image generation system 202 may determine the region of interest in the image based on the depth values. For example, mixed-reality image generation system 202 may determine whether the depth value associated with each node in the image is below a depth threshold such that the region of interest may include a region in which at least some of the depth values are below the depth threshold. For example, depth values below the depth threshold may indicate that the nodes associated with the depth values correspond to portions of anatomical object 216 that may be positioned proximate to imaging device 204 such that it may be desirable for a user to visualize that portion of anatomical object 216 (e.g., to perform a medical procedure). To illustrate, first depth d₁ of first surface point 506-1 is less than second depth d₂ of second surface point 506-2 such that the first depth value associated with first depth d₁ may be below the depth threshold while the second depth value associated with second depth d₂ may meet or exceed the depth threshold. Accordingly, mixed-reality image generation system 202 may determine the region of interest to include at least the first node associated with the first depth value below the depth threshold.

In some implementations, the region of interest may be determined based on depth values associated with each node in the image. For example, the region of interest may be sized to include at least some of the nodes and/or an area (e.g., a shape) surrounding at least some of the nodes in the image associated with depth values below the depth threshold. Additionally or alternatively, the region of interest may be determined based on a combination (e.g., an average, a mean, a median, etc.) of depth values associated with a plurality of nodes such that a single depth value may be associated with the plurality of nodes.

Mixed-reality image generation system 202 may further be configured to determine a single region of interest and/or multiple regions of interest in the image based on the depth values. For example, a single region of interest may be determined to include at least some of the nodes associated with depth values below the depth threshold, such as when the nodes are spaced within a predetermined distance of each other in the image. Additionally or alternatively, multiple regions of interest may be determined to include at least some of the nodes associated with depth values below the depth value, such as when the nodes are spaced outside of the predetermined distance of each other in the image. The one or more regions of interest may include at least a portion of one or more anatomical objects 216 depicted in the image.

In some implementations, mixed-reality image generation system 202 may update the depth values and/or the region of interest based on movement of anatomical object 216 (e.g., during a medical procedure). For example, mixed-reality image generation system 202 may track (e.g., using a SLAM algorithm) surface points 506 of anatomical object 216 such that mixed-reality image generation system 202 may update locations of the nodes in the image associated with surface points 506 based on movement of surface points 506. Mixed-reality image generation system 202 may further update the depth values associated with the updated locations of the nodes and determine whether the updated depth values are below the depth threshold. If some of the updated depth values transition (e.g., fall below and/or meet or exceed) the depth threshold, mixed-reality image generation system 202 may update the region of interest based on the transitions of the updated depth values. Additionally or alternatively, the depth values and/or the region of interest may be fixed for at least a period of time.

In some instances, one or more portions of anatomical object 216 may be obstructed from the field of view of imaging device 204 such that mixed-reality image generation system 202 may be configured to interpolate depth values for one or more nodes that may be associated with one or more surface points 506 on the portions of anatomical object 216 that may be obstructed. In such instances, mixed-reality image generation system 202 may be configured to associate a confidence value with each depth value representative of a confidence level of the depth value. The confidence value may be represented by any suitable metric, such as a discrete value (e.g., a level, a ratio, a percentage, etc.).

Based on the confidence values, mixed-reality image generation system 202 may be configured to determine a measurable area in the image such as by determining an area encompassing at least some the nodes associated with depth values having a confidence value that meets or exceeds a confidence threshold. For example, confidence values that meet or exceed the confidence threshold may indicate that the depth values of the respective nodes may be sufficiently accurate within the measurable area. Accordingly, mixed-reality image generation system 202 may determine the region of interest based on the depth values for nodes positioned within the measurable area.

FIG. 5B shows another illustrative implementation 508 of determining the region of interest based on a depth of anatomical object 216 such as relative to a tissue surface 510 (e.g., a surface of a skin layer, a surface of another anatomical object, etc.). To illustrate, mixed-reality image generation system 202 may determine a depth value associated with one or more nodes in the image representative of a depth D (e.g., depths D₁ to D₂) of surface points 506 on anatomical object 216 relative to tissue surface 510. For example, a first node may have a first depth value representative of a first depth D₁ of first surface point 506-1 relative to tissue surface 510 and a second node may have a second depth value representative of a second depth D₂ of second surface point 506-2 relative to tissue surface 510. The depth value may be represented by any suitable metric, such as a discrete value (e.g., a distance, a ratio, a percentage, etc.).

In some implementations, the depth values may be based on a depth D having any suitable orientation between surface points 506 and tissue surface 510, such as an orientation normal to exterior surface 502 of anatomical object 216 at surface points 506 and/or tissue surface 510, an orientation forming a shortest distance between surface points 506 and tissue surface 510, an orientation forming a farthest distance between surface points 506 and tissue surface 510, etc. In addition to or instead of surface points 506, the depth values may be associated with one or more other points (e.g., a centroid, etc.) of one or more anatomical objects 216.

Mixed-reality image generation system 202 may determine the region of interest based on the depth values. For example, mixed-reality image generation system 202 may determine whether the depth values associated with the nodes are below a depth threshold such that the region of interest may include a region in which at least some of the depth values are below the depth threshold. For example, depth values below the depth threshold may indicate that the nodes associated with the depth values correspond to portions of anatomical object 216 positioned proximate to tissue surface 510 such that it may be desirable for a user to visualize that portion of anatomical object 216 (e.g., to perform a medical procedure).

To illustrate, first depth D₁ of first surface point 506-1 is less than second depth D₂ of second surface point 506-2 such that the first depth value associated with first depth D₁ may be below the depth threshold while the second depth value associated with second depth D₂ may meet or exceed the depth threshold. Accordingly, mixed-reality image generation system 202 may determine the region of interest to include at least the first node associated with the first depth value below the depth threshold. In some implementations, mixed-reality image generation system 202 may determine one or more regions of interest to include at least some of the nodes associated with depth values below the depth threshold such that the one or more regions of interest include at least a portion of one or more anatomical objects 216 depicted in the image.

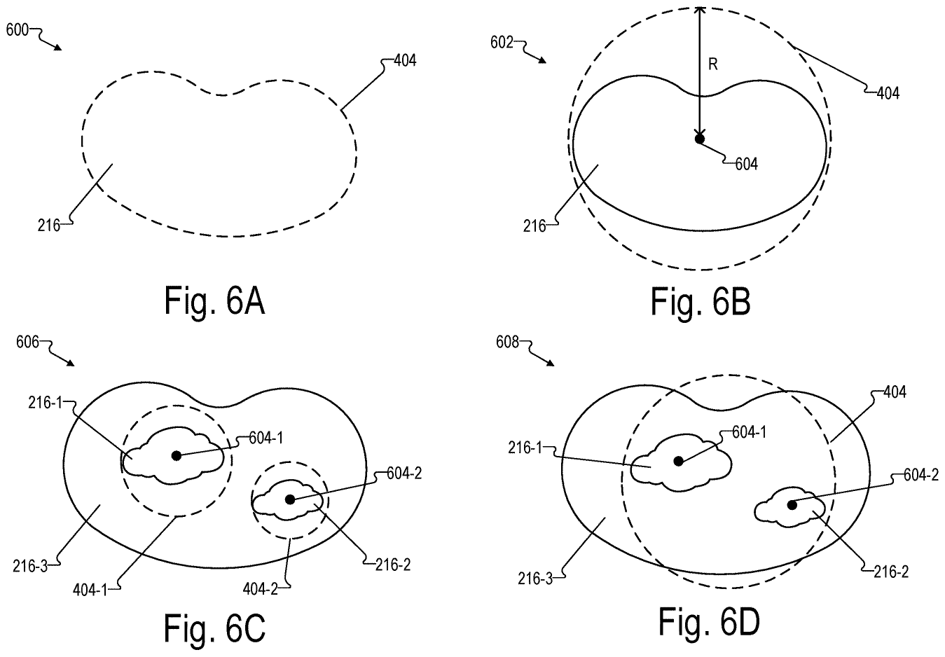

FIG. 6A shows an illustrative implementation 600 of region of interest 404 determined based on a size of one or more anatomical objects 216 depicted in the image (e.g., image 402). For example, mixed-reality image generation system 202 may determine a size of the one or more anatomical objects 216 such as by identifying (e.g., using machine learning algorithms, object identification, etc.) the one or more anatomical objects 216 and/or one or more features of the one or more anatomical objects 216 in the scene of the image and associating the identified anatomical objects 216 and/or features with pixels or voxels of the image. Mixed-reality image generation system 202 may further determine region of interest 404 based on the size of the identified anatomical objects 216 and/or features such as by sizing region of interest 404 to include an entirety and/or a portion of the pixels or voxels associated with the identified anatomical objects 216 and/or features. To illustrate, FIG. 6A shows region of interest 404 corresponding to the size of anatomical object 216. While implementation 600 shows region of interest 404 as including a boundary of anatomical object 216, region of interest 404 may additionally or alternatively include a shape (e.g., a circle, a square, etc.) encompassing the boundary of one or more anatomical objects 216.

FIG. 6B shows an illustrative implementation 602 of region of interest 404 determined based on a point 604 associated with one or more anatomical objects 216 in the image. For example, mixed-reality image generation system 202 may identify point 604 (e.g., a centroid, a point closest to imaging device 204 and/or tissue surface 510, a point farthest from imaging device 204 and/or tissue surface 510, etc.) associated with anatomical object 216. While implementation 602 shows point 604 associated with a single anatomical object 216, point 604 may additionally or alternatively be associated with multiple anatomical objects 216. To illustrate, point 604 may include a centroid of the multiple anatomical objects 216. In some implementations, point 604 may be identified by detecting a user input (e.g., using user input device 220) designating point 604 within the image and/or using machine learning algorithms.

Region of interest 404 may be determined to include a boundary spaced a select distance R away from point 604 in the image. As shown, region of interest 404 includes a circle having a radius of the select distance R about point 604 such that the select distance R is continuous about point 604. Additionally or alternatively, the select distance R may vary about point 604 such that other suitable shapes (e.g., a square, a triangle, freeform, etc.) may be used to form the boundary of region of interest 404 relative to point 604. In some implementations, the select distance R of the boundary of region of interest 404 may be adjustable (e.g., based on user input).

FIG. 6C shows an illustrative implementation 606 of a plurality of regions of interest 404 (e.g., regions of interest 404-1 to 404-2) determined based on a plurality of anatomical objects 216 (e.g., anatomical objects 216-1 to 216-3) depicted in the image. As shown, a first anatomical object 216-1 and a second anatomical object 216-2 are positioned within a third anatomical object 216-3. Moreover, a first point 604-1 is associated with first anatomical object 216-1 and a second point 604-2 is associated with second anatomical object 216-2. The plurality of regions of interest 404 may be determined to include a first region of interest 404-1 having a first boundary encompassing first point 604-1 associated with first anatomical object 216-1 and a second region of interest 404-2 having a second boundary encompassing second point 604-2 associated with second anatomical object 216-2. In some implementations, the boundary of each region of interest 404 may be based on a size of the respective anatomical object 216, a size of a feature of the respective anatomical object 216, a select distance away from the respective point 604, etc.

FIG. 6D shows an illustrative implementation 608 of a region of interest 404 determined based on a plurality of points 604 associated with one or more anatomical objects 216. For example, region of interest 404 may be determined to include a boundary encompassing both first point 604-1 associated with first anatomical object 216-1 and second point 604-2 associated with second anatomical object 216-2 in the image. While implementation 608 shows each point 604 associated with different anatomical objects 216, points 604 may additionally or alternatively be associated with the same anatomical object 216 (e.g., points 604 may be associated with different features of the same anatomical object 216). In some implementations, the boundary of region of interest 404 may be based on a size of the one or more anatomical objects 216, a size of one or more features of the one or more anatomical objects 216, a select distance away from the one or more points 604, etc.

Still other suitable techniques for determining region of interest 404 may be used. For example, region of interest 404 may be determined based on information associated with a medical procedure. To illustrate, the information may indicate one or more target anatomical objects 216 associated with the medical procedure such that mixed-reality image generation system 202 may identify the target anatomical objects 216 in the image and determine region of interest 404 to include the target anatomical objects 216.

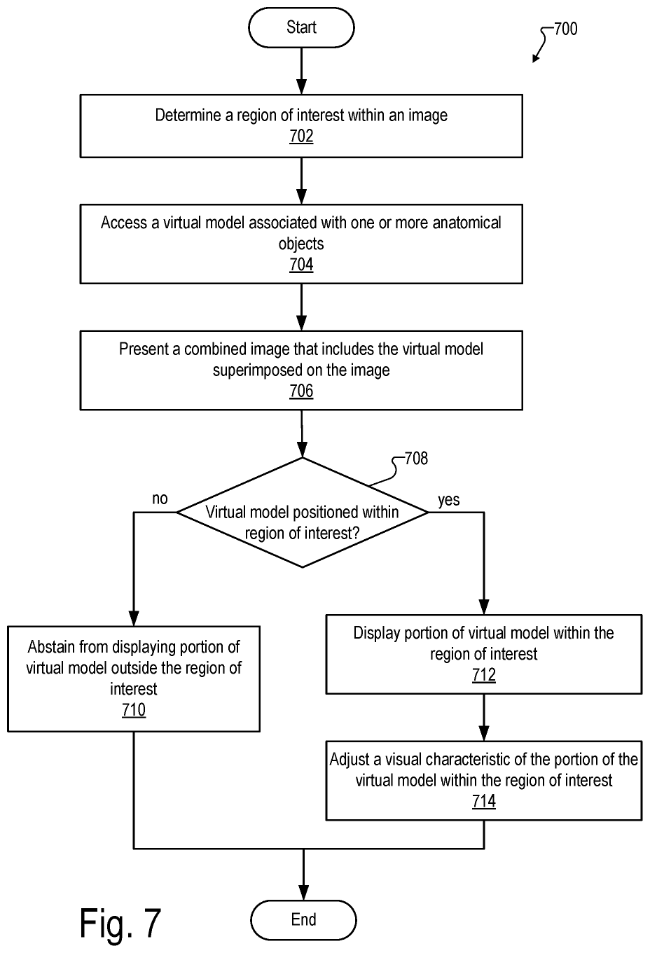

FIG. 7 shows another illustrative method 700 that may be performed by mixed-reality image generation system 202. While FIG. 7 illustrates exemplary operations according to one embodiment, other embodiments may omit, add to, reorder, and/or modify any of the operations shown in FIG. 7. Moreover, each of the operations depicted in FIG. 7 may be performed in any of the ways described herein.

As shown, mixed-reality image generation system 202 may, at operation 702, determine a region of interest (e.g., region of interest 404) within an image. For example, the image may depict a scene that includes one or more anatomical objects (e.g., anatomical objects 216) such that the region of interest may be determined to include at least a portion of the one or more anatomical objects in the scene. In some implementations, the determining the region of interest may be based on a depth of the one or more anatomical objects (e.g., relative to imaging device 204 and/or tissue surface 510), a size of the one or more anatomical objects, one or more points (e.g., points 604) associated with the one or more anatomical objects, detecting a user input designating the region of interest, etc.

Mixed-reality image generation system 202 may further, at operation 704, access a virtual model (e.g., virtual model 408) of the one or more anatomical objects and, at operation 706, present a combined image that includes the virtual model superimposed on the image. Mixed-reality image generation system 202 may further, at operation 708, determine whether the virtual model is positioned within the region of interest. For example, if a portion of the virtual model is not positioned within the region of interest (no, at operation 708), mixed-reality image generation system 202 may, at operation 710, direct the display device to abstain from displaying the portion of the virtual model outside the region of interest. Alternatively, if a portion of the virtual model is positioned within the region of interest (yes, at operation 708), mixed-reality image generation system 202 may, at operation 712, direct the display device to display the portion of the virtual model positioned within the region of interest.

In some implementations, mixed-reality image generation system 202 may further, at operation 714, adjust a visual characteristic (e.g., a transparency, a brightness, etc.) of the portion of the virtual model positioned within the region of interest. For example, one or more visual characteristics of the virtual model may be adjusted based on detecting a user input (e.g., using user input device 220) designating the visual characteristic and/or using machine learning algorithms. To illustrate, the one or more visual characteristics may be adjusted based on a depth of the one or more anatomical objects (e.g., relative to imaging device 204 and/or tissue surface 510), a size of the one or more anatomical objects, one or more points (e.g., points 604) associated with the one or more anatomical objects, etc. In some implementations, adjusting the one or more visual characteristics of the virtual model may improve a depth perception between the virtual model and the remaining portion of the image.

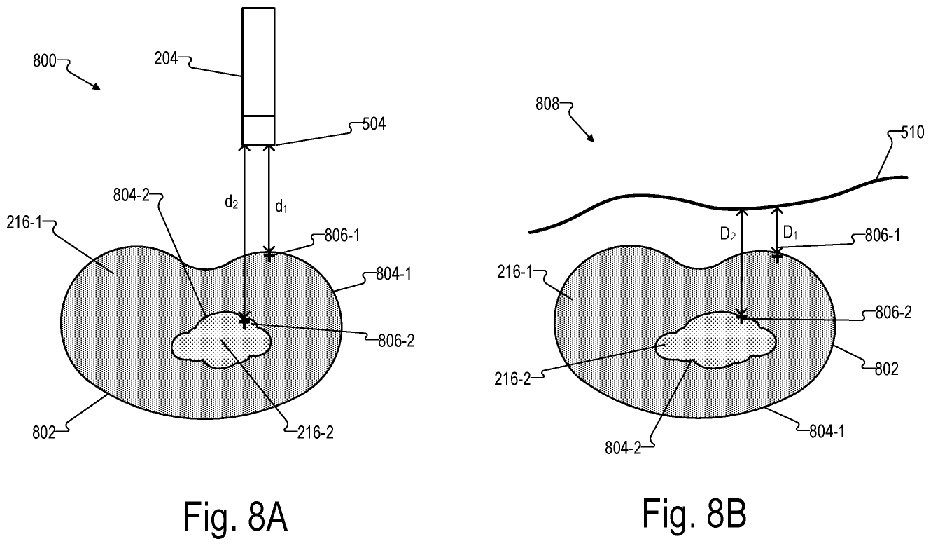

As an example, FIG. 8A shows an illustrative implementation 800 of a portion of a virtual model 802 that may be displayed within the region of interest in the image and adjusted based on a depth relative to imaging device 204. Virtual model 802 may implement or be similar to virtual model 408. As shown, virtual model 802 includes a first anatomical object 216-1 and a second anatomical object 216-2. In some implementations, anatomical objects 216 of virtual model 802 may be sized and/or oriented (e.g., based on user input) in the image to align virtual model 802 with corresponding anatomical objects 216 depicted in the image (e.g., as captured by imaging device 204). For example, a first exterior surface 804-1 of first anatomical object 216-1 of virtual model 802 may be aligned with the exterior surface (e.g., exterior surface 502) of a corresponding anatomical object 216 in the image and/or a second exterior surface 804-2 of second anatomical object 216-2 of virtual model 802 may be aligned with the exterior surface of a corresponding anatomical object 216 in the image.

In some implementations, anatomical objects 216 of virtual model 802 may be registered with the corresponding anatomical objects 216 in the image, such as by associating vertices 806 (e.g., vertices 806-1 to 806-1) of virtual model 802 with corresponding nodes representative of surface points (e.g., surface points 506) on anatomical objects 216 depicted in the image. This may allow the depth values associated with the nodes of anatomical objects 216 depicted in the image to be further associated with the corresponding vertices 806 of virtual model 802. For example, a first vertex 806-1 of virtual model 802 located on first exterior surface 804-1 of first anatomical object 216-1 may be associated with a first depth value of a first node representative of first depth d₁ relative to distal end 504 of imaging device 204. Likewise, a second vertex 806-2 of virtual model 802 located on second exterior surface 804-2 of second anatomical object 216-2 may be associated with a second depth value of a second node representative of second depth d₁ relative to distal end 504 of imaging device 204.

Based on the depth values, mixed-reality image generation system 202 may be configured to adjust the visual characteristic of virtual model 802 within the region of interest. For example, mixed-reality image generation system 202 may adjust the visual characteristic to cause the display device (e.g., display device 218) to display portions of virtual model 802 associated with depth values lower than the depth values of other portions of virtual model 802 as more opaque than the other portions of virtual model 802 (e.g., the transparency of virtual model 802 may decrease as the depth values decrease). For example, lower depth values may indicate that the portions of virtual model 802 may correspond to anatomical objects 216 positioned proximate to imaging device 204 such that it may be desirable for a user to more clearly visualize anatomical objects 216 positioned proximate to imaging device 204 than anatomical objects 216 positioned away from imaging device 204. To illustrate, the first depth d₁ associated with first vertex 806-1 of virtual model 802 is less than the second depth d₂ associated with second vertex 806-2 of virtual model 802 such that first anatomical object 216-1 associated with first vertex 806-1 is shown as more opaque than second anatomical object 216-2 associated with second vertex 806-2.

In some implementations, mixed-reality image generation system 202 may be configured to adjust the visual characteristic of virtual model 802 based on relative depth values such that portions of virtual model 802 associated with depth values lower than depth values of other portions of virtual model 802 may be shown as more opaque. Additionally or alternatively, the visual characteristic may be adjusted based on absolute depth values such that portions of virtual model 802 associated with depth values below a depth threshold may be shown as opaque while portions of virtual model 802 associated with depth values that meet or exceed the depth threshold may be shown as more transparent.

In the illustrated implementation, the adjustment of the visual characteristic of virtual model 802 is applied consistently across each anatomical object 216 of virtual model 802 such that each anatomical object 216 is shown at a constant opacity (e.g., the entire first anatomical object 216-1 is shown as more opaque than the entire second anatomical object 216-2). Additionally or alternatively, the visual characteristic of virtual model 802 may be adjusted to vary across one or more anatomical objects 216 of virtual model 802. For example, the transparency of portions of virtual model 802 may increase along anatomical objects 216 of virtual model 802 as the depth values of the portions of virtual model 802 increase (e.g., away from imaging device 204).

In some implementations, mixed-reality image generation system 202 may update the visual characteristic adjustment of virtual model 802 (e.g., based on movement of the corresponding anatomical object 216 in the image, user input, etc.). For example, mixed-reality image generation system 202 may update the visual characteristic adjustment based on a change of the depth values associated with virtual model 802 such that portions of virtual model 802 may be shown as more opaque if the depth values decrease and/or more transparent if the depth values increase. Additionally or alternatively, the adjustment of the visual characteristic may be fixed for at least a period of time. In some implementations, the visual characteristic adjustment may be further adjusted, such as based on a user input designating further adjustment of the visual characteristic after the visual characteristic has been adjusted based on the depth of the one or more anatomical objects 216.

FIG. 8B shows an illustrative implementation 808 of adjusting the visual characteristic of virtual model 802 based on a depth relative to tissue surface 510. As shown, a first vertex 806-1 located on a first exterior surface 804-1 of first anatomical object 216-1 included in virtual model 802 may be associated with a depth value representative of a first depth D₁ relative to tissue surface 510 and a second vertex 806-2 located on a second exterior surface 804-2 of second anatomical object 216-2 included in virtual model 802 may be associated with a depth value representative of a second depth D₂ relative to tissue surface 510. In some implementations, mixed-reality system 202 may determine which tissue surface 510 is used for determining the depth values (e.g., a tissue surface 510 closest to a respective vertex 806, a tissue surface 510 normal to a respective vertex 806, etc.). Additionally or alternatively, a user may designate which tissue surface 510 is used for determining the depth values (e.g., using user input device 220).

Based on the depth values, mixed-reality image generation system 202 may be configured to adjust the visual characteristic of virtual model 802 within the region of interest. For example, mixed-reality image generation system 202 may adjust the visual characteristic to cause the display device (e.g., display device 218) to display portions of virtual model 802 associated with depth values lower than the depth values of other portions of virtual model 802 as more opaque than the other portions of virtual model 802 (e.g., the transparency of virtual model 802 may decrease as the depth values decrease). For example, lower depth values may indicate that the portions of virtual model 802 may correspond to anatomical objects 216 positioned proximate to tissue surface 510 such that it may be desirable for a user to more clearly visualize anatomical objects 216 positioned proximate to tissue surface 510 than anatomical objects 216 positioned away from tissue surface 510. To illustrate, the first depth D₁ associated with first vertex 806-1 of virtual model 802 is less than the second depth D₂ associated with second vertex 806-2 of virtual model 802 such that first anatomical object 216-1 associated with first vertex 806-1 is shown as more opaque than second anatomical object 216-2 associated with second vertex 806-2.

In some implementations, the visual characteristic of virtual model 802 may be adjusted based on a single depth value (e.g., associated with one or more vertices 806 of virtual model 802 that may correspond to a centroid of one or more anatomical object 216, a closest point to tissue surface 510, a farthest point to tissue surface 510, etc.). This may allow the visual characteristic may be adjusted consistently across each anatomical object 216 of virtual model 802 such that each anatomical object 216 may be shown at a constant opacity (e.g., the entire first anatomical object 216-1 is shown as more opaque than the entire second anatomical object 216-2). Additionally or alternatively, the visual characteristic of virtual model 802 may be adjusted based on a plurality of depth values associated with various portions of virtual model 802 such that the transparency of the various portions of virtual model 802 may increase along anatomical objects 216 of virtual model 802 as the depth values of the various portions of virtual model 802 increase. In some implementations, the visual characteristic of virtual model 802 may be scaled (e.g., based on a depth relative to imaging device and/or tissue surface 510) such that a region of interest in the image may not be explicitly determined. Still other depth-based techniques for adjusting the visual characteristic of virtual model 802 may be used.

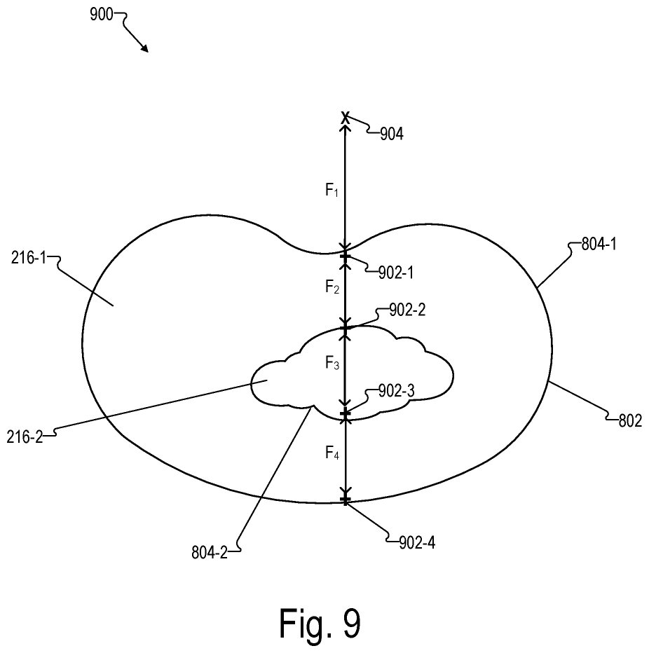

For example, FIG. 9 shows an illustrative implementation 900 for adjusting the visual characteristic of virtual model 802 based on a plurality of fragmented depth values associated with one or more vertices 902 (e.g., vertices 902-1 to 902-4) of virtual model 802. For example, vertices 902 of virtual model 802 may be identified at exterior surfaces 804 of anatomical objects 216 included in virtual model 802 that may be aligned with a reference location 904 (e.g., associated with imaging device 204 and/or tissue surface 510). Fragmented depth values may be associated with the identified vertices 902 based on one or more fragmented depths F (e.g., fragmented depths F₁ to F₄) that may represent a depth from the identified vertex 902 to reference location 904 and/or another identified vertex 902. Accordingly, the visual characteristic of virtual model 802 may be adjusted based on the fragmented depth values associated with vertices 902 of virtual model 802.

To illustrate, implementation 900 includes a first vertex 902-1 located on first exterior surface 804-1 of first anatomical object 216-1, a second vertex 902-2 located on second exterior surface 804-2 of second anatomical object 216-2, a third vertex 902-3 located on second exterior surface 804-2 of second anatomical object 216-2 opposite second vertex 902-2, and a fourth vertex 902-4 located on first exterior surface 804-1 of first anatomical object 216-1 opposite first vertex 902-1 such that each vertex 902 is aligned with reference location 904.

Based on the identified vertices 902, mixed-reality image generation system 202 may determine fragmented depth values associated with the identified vertices 902. For example, a first fragmented depth value may be associated with first vertex 902-1 (e.g., a vertex 902 closest to reference location 904) based on a first fragmented depth F₁ from first vertex 902-1 to reference location 904. In some implementations, the depth values associated with the remaining vertices 902 may be based on a single fragmented depth F and/or a combination of fragmented depths F (e.g., such that the fragmented depths F may build on each other). To illustrate, a second fragmented depth value may be associated with second vertex 902-2 based on a second fragmented depth F₂ from second vertex 902-2 to first vertex 902-1 and/or a combination of first fragmented depth F₁ and second fragmented depth F₂. A third fragmented depth value may be associated with third vertex 902-3 based on a third fragmented depth F₃ from third vertex 902-3 to second vertex 902-2 and/or a combination of first fragmented depth F₁, second fragmented depth F₂, and third fragmented depth F₃. A fourth fragmented depth value may be associated with fourth vertex 902-4 based on a fourth fragmented depth F₄ from fourth vertex 902-4 to third vertex 902-3 and/or a combination of first fragmented depth F₁, second fragmented depth F₂, third fragmented depth F₃, and fourth fragmented depth F₄.

Mixed-reality image generation system 202 may adjust the visual characteristic of virtual model 802 based on the fragmented depth values associated with vertices 902 of virtual model 802. For example, portions of virtual model 802 may be displayed as more transparent as the fragmented depth values associated with the portions of virtual model 802 increase and/or as a number of fragmented depths F used to determine the fragmented depth values increases. To illustrate, the display of virtual model 802 may increase in transparency from first vertex 902-1 to fourth vertex 902-4.

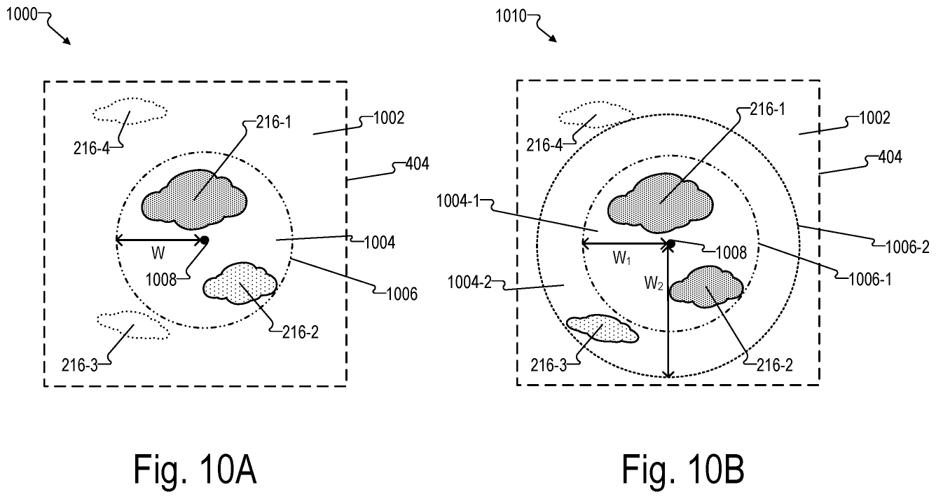

FIG. 10A shows an illustrative implementation 1000 of a portion of a virtual model 1002 displayed within a region of interest 404 that may be adjusted within a display region 1004 positioned within region of interest 404. Virtual model 1002 may implement or be similar to virtual model 408 and/or virtual model 802. As shown, virtual model 1002 includes a first anatomical object 216-1 and a second anatomical object 216-2 positioned within display region 1004 as well as a third anatomical object 216-3 and a fourth anatomical object 216-4 positioned outside of display region 1004.

In some implementations, display region 1004 may be formed by a display boundary 1006 associated with a point 1008. For example, point 1008 may be associated with virtual model 1002 and/or region of interest 404. To illustrate, point 1008 may be identified as a centroid of region of interest 404, a centroid of one or more anatomical objects 216 depicted in region of interest 404, a centroid of one or more anatomical objects 216 of virtual model 1002, etc. In some implementations, point 1008 may be identified by detecting a user input (e.g., using user input device 220) designating point 1008 within the image and/or using machine learning algorithms.

Display boundary 1006 may be spaced a select width W away from point 1008 within region of interest 404. As shown, display boundary 1006 includes a circle having a radius of the select width W about point 1008 such that the select width W is continuous about point 1008. Additionally or alternatively, the select width W may vary about point 1008 such that other suitable shapes (e.g., a square, a triangle, freeform, etc.) may be used to form display boundary 1006 relative to point 1008. In some implementations, the select width W of display boundary 1006 may include a predetermined distance, such as designated by a user input. Additionally or alternatively, the select width W of display boundary 1006 may be adjustable (e.g., based on user input).

In some implementations, one or more visual characteristics of virtual model 1002 may be adjusted within display region 1004. For example, the transparency of anatomical objects 216 of virtual model 1002 may be adjusted within display region 1004. To illustrate, first anatomical object 216-1 of virtual model 1002 is shown as being more opaque than second anatomical object 216-2 of virtual model 1002 within display region 1004. In some implementations, the visual characteristic of virtual model 1002 may be adjusted based on a distance of portions of virtual model 1002 relative to point 1008 (e.g., portions of virtual model 1002 positioned closer to point 1008 may be more opaque than other portions of virtual model 1002 positioned away from point 1008). Additionally or alternatively, the visual characteristic of virtual model 1002 may be adjusted based on a depth of one or more anatomical objects (e.g., relative to imaging device 204 and/or tissue surface 510), a size of one or more anatomical objects, etc.

In some implementations, portions of virtual model 1002 positioned outside of display region 1004, such as third anatomical object 216-3 and fourth anatomical object 216-4, may be displayed as transparent (e.g., relative to first anatomical object 216-1 and second anatomical object 216-2). Alternatively, portions of virtual model 1002 positioned within display region 1004, such as first anatomical object 216-1 and second anatomical object 216-2, may be displayed as opaque while the visual characteristic of other portions of virtual model 1002 positioned outside of display region 1004, such as third anatomical object 216-3 and fourth anatomical object 216-4, may be adjusted (e.g., to increase the transparency of portions of virtual model 1002 as the portions of virtual model 1002 are positioned away from display region 1004 and/or point 1008).

FIG. 10B shows an illustrative implementation 1010 of a portion of a virtual model 1002 displayed within a region of interest 404 that may be adjusted within a plurality of display regions 1004 (e.g., display regions 1004-1 to 1004-2) positioned within region of interest 404. As shown, the plurality of display regions 1004 include a first display region 1004-1 formed by a first display boundary 1006-1 spaced a first width W₁ away from point 1008 and a second display region 1004-2 formed by a second display boundary 1006-2 spaced a second width W₂ away from point 1008 that is greater than first width W₁. Each width W of display boundaries 1006 may be continuous about point 1008 and/or may vary about point 1008. In some implementations, each width W of display boundaries 1006 may include a predetermined distance, such as designated by a user input. Additionally or alternatively, each width W of display boundaries 1006 may be adjustable (e.g., based on user input).

In some implementations, one or more visual characteristics of virtual model 1002 may be adjusted between the plurality of display regions 1004. For example, the transparency of anatomical objects 216 of virtual model 1002 may be adjusted between first display region 1004-1 and second display region 1004-2. To illustrate, first anatomical object 216-1 and second anatomical object 216-2 of virtual model 1002 are positioned within first display boundary 1006-1 of first display region 1004-1 such that first anatomical object 216-1 and second anatomical object 216-2 may be shown as opaque. Third anatomical object 216-3 of virtual model 1002 is positioned between first display boundary 1006-1 and second display boundary 1006-2 within second display region 1004-2 such that the visual characteristic of third anatomical object 216-3 may be adjusted. For example, third anatomical object 216-3 may be displayed as more transparent than first anatomical object 216-1 and second anatomical object 216-2 within first display region 1004-1 (e.g., based on a distance of third anatomical object 216-2 relative to point 1008, a depth of third anatomical object 216-3, a size of third anatomical object 216-3, etc.). Fourth anatomical object 216-4 of virtual model 1002 is positioned outside of second display boundary 1006-2 such that fourth anatomical object 216-4 may be displayed as transparent (e.g., relative to third anatomical object 216-3).

In some implementations, each display boundary 1006 of each display region 1004 may include a shape (e.g., a circle, a square, etc.) while the display region 1004 is fully positioned within region of interest 404. Additionally, width W of the display boundary 1006 may be increased toward a boundary of region of interest 404 such that the shape of the display boundary 1006 may be modified as the display boundary 1006 meets the boundary of region of interest 404 to confine the display region 1004 within region of interest 404. For example, second display boundary 1006-2 is shown as a circle about point 1008. As the width W₂ of second display boundary 1006-2 increases, the edges of the second display boundary 1006-2 may flatten to the square shape of region of interest 404 to confine second display region 1004-2 to region of interest 404.

Still other suitable techniques for adjusting one or more visual characteristics of a virtual model may be used. For example, one or more visual characteristics may be adjusted based on information associated with a medical procedure. To illustrate, the information may indicate one or more target anatomical objects associated with the medical procedure such that mixed-reality image generation system 202 may adjust one or more visual characteristics of the virtual model based on the one or more target anatomical objects (e.g., the one or more target anatomical objects included in the virtual model may be displayed as more opaque than other anatomical objects included in the virtual model).

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (“DRAM”), which typically constitutes a main memory. Common forms of computer-readable media include, for example, a disk, hard disk, magnetic tape, any other magnetic medium, a compact disc read-only memory (“CD-ROM”), a digital video disc (“DVD”), any other optical medium, random access memory (“RAM”), programmable read-only memory (“PROM”), electrically erasable programmable read-only memory (“EPROM”), FLASH-EEPROM, any other memory chip or cartridge, or any other tangible medium from which a computer can read.

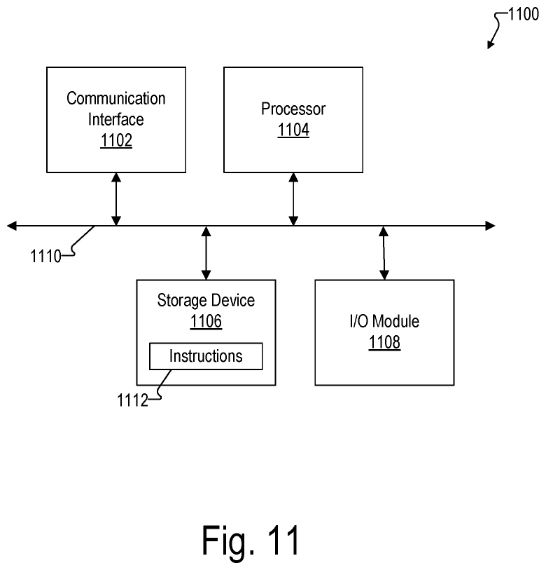

FIG. 11 shows an illustrative computing device 1100 that may be specifically configured to perform one or more of the processes described herein. Any of the systems, computing devices, and/or other components described herein may be implemented by computing device 1100.

As shown in FIG. 11, computing device 1100 may include a communication interface 1102, a processor 1104, a storage device 1106, and an input/output (“I/O”) module 1108 communicatively connected one to another via a communication infrastructure 1110. While an illustrative computing device 1100 is shown in FIG. 11, the components illustrated in FIG. 11 are not intended to be limiting. Additional or alternative components may be used in other embodiments. Components of computing device 1100 shown in FIG. 11 will now be described in additional detail.

Communication interface 1102 may be configured to communicate with one or more computing devices. Examples of communication interface 1102 include, without limitation, a wired network interface (such as a network interface card), a wireless network interface (such as a wireless network interface card), a modem, an audio/video connection, and any other suitable interface.

Processor 1104 generally represents any type or form of processing unit capable of processing data and/or interpreting, executing, and/or directing execution of one or more of the instructions, processes, and/or operations described herein. Processor 1104 may perform operations by executing computer-executable instructions 1112 (e.g., an application, software, code, and/or other executable data instance) stored in storage device 1106.

Storage device 1106 may include one or more data storage media, devices, or configurations and may employ any type, form, and combination of data storage media and/or device. For example, storage device 1106 may include, but is not limited to, any combination of the non-volatile media and/or volatile media described herein. Electronic data, including data described herein, may be temporarily and/or permanently stored in storage device 1106. For example, data representative of computer-executable instructions 1112 configured to direct processor 1104 to perform any of the operations described herein may be stored within storage device 1106. In some examples, data may be arranged in one or more databases residing within storage device 1106.

I/O module 1108 may include one or more I/O modules configured to receive user input and provide user output. I/O module 1108 may include any hardware, firmware, software, or combination thereof supportive of input and output capabilities. For example, I/O module 1108 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1108 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1108 is configured to provide graphical data to a display for presentation to a user. The graphical data may be representative of one or more graphical user interfaces and/or any other graphical content as may serve a particular implementation.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A computer-assisted medical system comprising:
a repositionable manipulator arm configured to be coupled to an imaging device, the imaging device configured to capture an image depicting a scene that includes one or more anatomical objects positioned within a field of view of the imaging device; and
a computing device communicatively coupled with the imaging device and configured to:
determine a region of interest within the image;
access a virtual model associated with the one or more anatomical objects; and
present a combined image that includes the virtual model superimposed on the image by directing a display device to:
display, within the combined image, a first portion of the virtual model that is positioned within the region of interest of the image, and
abstain from displaying, within the combined image, a second portion of the virtual model that is positioned outside the region of interest of the image.

2. The computer-assisted medical system of claim 1, wherein the computing device is further configured to cause the repositionable manipulator arm to position the field of view of the imaging device to include the one or more anatomical objects.

3. The computer-assisted medical system of claim 1, further comprising a second repositionable manipulator arm configured to be coupled to an instrument, wherein the computing device is further configured to cause the second repositionable manipulator arm to move the instrument within the field of view of the imaging device to manipulate the one or more anatomical objects.

4. The computer-assisted medical system of claim 1, wherein the determining the region of interest is based on a depth of the one or more anatomical objects relative to the imaging device.

5. The computer-assisted medical system of claim 1, wherein the determining the region of interest is based on a depth of the one or more anatomical objects relative to a tissue surface.

6. The computer-assisted medical system of claim 1, wherein the determining the region of interest is based on a size of the one or more anatomical objects.

7. The computer-assisted medical system of claim 1, wherein the determining the region of interest includes determining a boundary of the region of interest spaced a select distance away from a point associated with the one or more anatomical objects.

8. The computer-assisted medical system of claim 7, wherein the point is a centroid identified based on the one or more anatomical objects.

9. The computer-assisted medical system of claim 7, wherein the point is designated on the one or more anatomical objects by a user input.

10. The computer-assisted medical system of claim 7, wherein the select distance of the boundary is adjustable.

11. The computer-assisted medical system of claim 1, wherein the region of interest comprises a first sub-region of interest and a second sub-region of interest, wherein the determining the region of interest comprises:
determining the first sub-region of interest by determining a first boundary encompassing a first point associated with a first anatomical object; and
determining the second sub-region of interest by determining a second boundary encompassing a second point associated with a second anatomical object.

12. The computer-assisted medical system of claim 1, wherein the determining the region of interest includes determining a boundary encompassing a first point associated with a first anatomical object and a second point associated with a second anatomical object.

13. The computer-assisted medical system of claim 1, wherein the determining the region of interest is based on detecting a user input designating the region of interest.

14. The computer-assisted medical system of claim 1, wherein the determining the region of interest includes identifying the one or more anatomical objects.

15. The computer-assisted medical system of claim 1, wherein the displaying the first portion of the virtual model includes displaying an outline of the region of interest.

16. The computer-assisted medical system of claim 1, wherein the displaying the first portion of the virtual model includes adjusting a visual characteristic of the first portion of the virtual model within the region of interest.

17. The computer-assisted medical system of claim 16, wherein the adjusting the visual characteristic is based on a depth of the one or more anatomical objects relative to the imaging device.

18. The computer-assisted medical system of claim 16, wherein the adjusting the visual characteristic is based on a depth of the one or more anatomical objects relative to a tissue surface.

19. The computer-assisted medical system of claim 18, wherein the depth of the one or more anatomical objects relative to the tissue surface is based on a single depth value.

20. The computer-assisted medical system of claim 18, wherein the depth of the one or more anatomical objects relative to the tissue surface is based on a plurality of fragmented depth values.

21. The computer-assisted medical system of claim 16, wherein the adjusting the visual characteristic is based on a size of the one or more anatomical objects.

22. The computer-assisted medical system of claim 16, wherein the visual characteristic is adjustable within a display region positioned within the region of interest, wherein the display region includes a display boundary spaced a select distance away from a point associated with the one or more anatomical objects.

23. The computer-assisted medical system of claim 22, wherein the point is a centroid identified based on the one or more anatomical objects.

24. The computer-assisted medical system of claim 22, wherein the point is designated on the one or more anatomical objects by a user input.

25. The computer-assisted medical system of claim 22, wherein the select distance of the display boundary is adjustable.

26. The computer-assisted medical system of claim 22, wherein the select distance of the display boundary is a predetermined distance.

27. The computer-assisted medical system of claim 22, wherein the select distance of the display boundary is designated by a user input.

28. The computer-assisted medical system of claim 22, wherein the display boundary includes a first display boundary spaced a first distance away from a point associated with the one or more anatomical objects and a second display boundary spaced a second distance away from the point that is greater than the first distance, wherein the visual characteristic is adjustable between the first display boundary and the second display boundary.

29. The computer-assisted medical system of claim 28, wherein a select one or both of the first distance or the second distance is adjustable.

30. The computer-assisted medical system of claim 22, wherein the display boundary includes a shape while the display boundary is positioned within the region of interest.

31. The computer-assisted medical system of claim 30, wherein the display boundary is confined to the region of interest such that the shape of the display boundary is modified as the display boundary meets a boundary of the region of interest.

32. The computer-assisted medical system of claim 16, wherein the adjusting the visual characteristic is based on detecting a user input designating the visual characteristic.

33. The computer-assisted medical system of claim 1, wherein the presenting the combined image includes detecting that the first portion of the virtual model is repositioned to be outside the region of interest, and in response, abstaining from displaying the first portion.

34. The computer-assisted medical system of claim 1, wherein the presenting the combined image includes detecting that the second portion of the virtual model is repositioned to be inside the region of interest, and in response, displaying the second portion.

35. The computer-assisted medical system of claim 1, wherein the virtual model is based on preoperative imagery of the one or more anatomical objects.

36. A system comprising:
a memory storing instructions; and
one or more processors communicatively coupled to the memory and configured to execute the instructions to perform a process comprising:
determining a region of interest within an image depicting a scene that includes one or more anatomical objects;
accessing a virtual model associated with the one or more anatomical objects; and
presenting a combined image that includes the virtual model superimposed on the image, the presenting comprising directing a display device to:
display, within the combined image, a first portion of the virtual model that is positioned within the region of interest of the image, and
abstain from displaying, within the combined image, a second portion of the virtual model that is positioned outside the region of interest of the image.

37. The system of claim 36, wherein the determining the region of interest is based on a depth of the one or more anatomical objects relative to an instrument.

38. The system of claim 36, wherein the determining the region of interest is based on a depth of the one or more anatomical objects relative to a tissue surface.

39. The system of claim 36, wherein the determining the region of interest comprises determining a depth of the one or more anatomical objects by generating a depth map of the scene.

40. The system of claim 36, wherein the determining the region of interest is based on a size of the one or more anatomical objects.

41. The system of claim 36, wherein the determining the region of interest comprises determining a boundary of the region of interest spaced a select distance away from a point associated with the one or more anatomical objects.

42. The system of claim 36, wherein the determining the region of interest is based on detecting a user input designating the region of interest.

43. The system of claim 36, wherein the displaying the first portion of the virtual model includes adjusting a visual characteristic of the first portion of the virtual model within the region of interest.

44. The system of claim 43, wherein the adjusting the visual characteristic is based on a depth of the one or more anatomical objects relative to an instrument.

45. The system of claim 43, wherein the adjusting the visual characteristic is based on a depth of the one or more anatomical objects relative to a tissue surface.

46. The system of claim 43, wherein the adjusting the visual characteristic is based on a size of the one or more anatomical objects.

47. The system of claim 43, wherein the visual characteristic is adjustable within a display region positioned within the region of interest, wherein the display region includes a display boundary spaced a select distance away from a point associated with the one or more anatomical objects.

48. The system of claim 47, wherein the display boundary includes a first display boundary spaced a first distance away from a point associated with the one or more anatomical objects and a second display boundary spaced a second distance away from the point that is greater than the first distance, wherein the visual characteristic is adjustable between the first display boundary and the second display boundary.

49. The system of claim 43, wherein the adjusting the visual characteristic is based on detecting a user input designating the visual characteristic.

50. The system of claim 36, wherein the presenting the combined image includes detecting that the first portion of the virtual model is repositioned to be outside the region of interest, and in response, abstaining from displaying the first portion.

51. The system of claim 36, wherein the presenting the combined image includes detecting that the second portion of the virtual model is repositioned to be inside the region of interest, and in response, displaying the second portion.

52. A method comprising:
determining a region of interest within an image depicting a scene that includes one or more anatomical objects;
accessing a virtual model associated with the one or more anatomical objects; and
presenting a combined image that includes the virtual model superimposed on the image, the presenting comprising directing a display device to:
display, within the combined image, a first portion of the virtual model that is positioned within the region of interest of the image, and
abstain from displaying, within the combined image, a second portion of the virtual model that is positioned outside the region of interest of the image.

53. The method of claim 52, wherein the determining the region of interest is based on a depth of the one or more anatomical objects.

54. The method of claim 52, wherein the determining the region of interest is based on a size of the one or more anatomical objects.

55. The method of claim 52, wherein the determining the region of interest comprises determining a boundary of the region of interest spaced a select distance away from a point associated with the one or more anatomical objects.

56. The method of claim 52, wherein the determining the region of interest is based on detecting a user input designating the region of interest.

57. The method of claim 52, wherein the displaying the first portion of the virtual model includes adjusting a visual characteristic of the first portion of the virtual model within the region of interest.

58. The method of claim 52, wherein the presenting the combined image includes detecting that the first portion of the virtual model is repositioned to be outside the region of interest, and in response, abstaining from displaying the first portion.

59. The method of claim 52, wherein the presenting the combined image includes detecting that the second portion of the virtual model is repositioned to be inside the region of interest, and in response, displaying the second portion.

60. A non-transitory computer-readable medium storing instructions that, when executed, direct a processor of a computing device to perform a process comprising:
determining a region of interest within an image depicting a scene that includes one or more anatomical objects;
accessing a virtual model associated with the one or more anatomical objects; and
presenting a combined image that includes the virtual model superimposed on the image, the presenting comprising directing a display device to:
display, within the combined image, a first portion of the virtual model that is positioned within the region of interest of the image, and
abstain from displaying, within the combined image, a second portion of the virtual model that is positioned outside the region of interest of the image.
